(19) United States
(12) Patent Application Publication
(10) Pub. No.: US 2007/0020340 A1
(43) Pub. Date: Jan. 25, 2007

(54) Fish oil products for reducing cholesterol, low density lipoprotein, and hypertension

(76) Inventors: David Rubin, San Diego, CA (US); Eyal Rubin, San Diego, CA (US)

Correspondence Address: BROWDY AND NEIMARK, P.L.L.C.;624 NINTH STREET, NW, SUITE 300, WASHINGTON, DC, 20001-5303, US

(21) Appl. No.: 11/188,124

(22) Filed: Jul. 25, 2005

(51) Int. Cl. A61K 36/02; A61K 35/60; A61K 31/22; A61K 31/05; A01N 31/08

(52) U.S. Cl. 424/523; 424/195.17; 424/776; 514/547; 514/731; 514/706

(57) ABSTRACT

A composition containing EPA and EHA, preferably derived from fish oil, in combination with at least one of oleuropein, allicin, and policosanol having anti-inflammatory, anti-thrombotic, immunomodulatory activity as well as activity to lower triglycerides and low density lipoprotein, to increase high density lipoprotein, and to lower both systolic and diastolic blood pressure.

### FIELD OF THE INVENTION

The present invention relates to fish oil products that can be used to treat elevated LDL-cholesterol, elevated triglycerides, insufficient HDL-cholesterol and hypertension.

### BACKGROUND OF THE INVENTION

For all humans, an appreciable reduction in the levels of total cholesterol, triglycerides, and low-density lipoprotein (LDL) in their blood serum, is known to be important for reducing the risk of cardiac and cerebro-vascular diseases. It is also known that affecting an increase in the levels of high-density lipoprotein (HDL) also provides a significant decrease to the risk of cardiac and cerebro-vascular diseases. Many people are also hypertensive, so reducing blood pressure will also reduce their risk of cardiac and cerebro-vascular diseases.

Cardiovascular and cerebro-vascular diseases resulting from the buildup of arterial plaque is known to be a leading cause of illness and death in humans. Arterial plaque is caused by precipitous material formed chiefly of oxidized low density lipoprotein (O-LDL). The buildup of plaque due to O-LDL in the arteries is understood to be a factor in all-ischemic diseases. Free radical oxidants, many of which come from naturally occurring sources such as sun exposure, metabolism of certain nutrients, exercise, or are otherwise often observed in persons suffering from diabetes and hypertension, act to oxidize LDL into its deleterious form O-LDL. In contrast, high density lipoprotein (HDL) in the body is understood to have beneficial health effects. Specifically, HDL is known to remove cholesterol from the blood vessels into the liver to be metabolized. HDL is able to absorb plaque material and may thus directly reduce the amount of arterial plaque.

Essential fatty acids are naturally occurring unsaturated fatty acids with a chain length of 18, 20 or 22 carbon atoms. These essential fatty acids cannot be synthesized by the body, so that dietary intake of these essential fatty acids is required. Two fatty acids that fall within the family of essential fatty acids are eicosapentaenoic acid (EPA) and docosahexaenoic acid (DHA), both of which are commonly found in fish oils and algae.

Epidemiological observations indicate that fish oils may reduce platelet aggregation and serum triglycerides, which may reduce the risk of myocardial infarction, hypertension, atherosclerosis, and certain types of cancer (Gerster, Internat. J. Vit Nutr Res 65: 3-20, 1994). Specifically, it has been shown that EPA and DHA derived from fish oils or algae play important structural roles in membranes of most cells, and influence the fluidity of the cell membranes expressed by decreased whole-blood viscosity and increased erythrocyte flexibility and deformability. In addition, EPA and DHA are known precursors of eicosanoids, a class of compound, which includes prostanoids such as prostaglandin and thromboxanes, leukotrienes, and hydroxy fatty acids. Eicosanoids are known to affect platelet aggregation, permeability and tone of the blood vessel wall, blood pressure, and inflammatory and immune reactions.

Supplemental fish oils, which contain both EPA and DHA, are known to have a triglyceride lowering activity, as well as anti-inflammatory, anti thrombotic, anti-arrhythmic properties and immunomodulatory action.

The triglyceride lowering effect of DHA and EPA results from inhibition of lipogenesis and stimulation of fatty acids oxidation in the liver. The anti-inflammatory properties of EPA and DHA are mainly the result of their competitive inhibition of the enzymes cyclooxygenase and lipoxygenase. They compete with arachidonic acid and reduce the production of prostaglandin E2 and leukotriene B4 that are responsible for the inflammatory reactions.

Unfortunately, most studies, including some made by the present inventors, show a substantial increase of LDL (low density lipoprotein) when a patient is treated with fish oil.

Turner et al., Int J Vitam Nutr Res 75(1) 61-70, January, 2005, investigated the antioxidant and cellular activity of the olive oil phenols, including oleuropein, tyrosol, hydroxytyrosol, and homovanillic alcohol. Antioxidant assays indicated that homovanillic alcohol was a significantly more potent antioxidant than the other phenolics.

Miles et al. reported in Nutrition 21(3):389-94 March 2005, that oleuropein glycoside and carreic acid decreased the concentration of interleukin-1-beta.

Masella et al., in J Nutr 134(4):785-791, 204, investigated the mechanisms underlying the protective effect exerted by extra virgin olive oil phenols, including oleuropein, on LDL oxidation mediated by murine J774A.1 macrophage-like cells. The biophenols were added to the cells with LDL and left in the medium during the entire experimental period. These antioxidants had the following effects:
1. completely prevented the 1774 A.1-mediated oxidation of LDL;
2. counteracted the time-dependent variations in intracellular redox balance, inhibiting the production Of O⁻² and H₂O₂ and the decrease in glutathione content;
3. restored glutathibne reductase and peroxidase activities; and
4. restored the mRNA expression of gamma-glutamylcysteine synthetase, glutathione reductase, and peroxidase activities to control values.

Allicin, an active constituent of garlic, has been reported to affect the serum lipid profile. Eilat et al., in Coron Artery Dis 6(12): 985-990, 1995, fed rabbits a cholesterol-rich diet, and ten rabbits received freshly produced allicin. It was found that allicin has a beneficial effect on the serum lipid profile in hyperlipidemic rabbits.

Hsia et al., in U.S. Pat. No. 6,326,031, disclose a nutritional supplement for decreasing cholesterol and triglycerides levels. This composition contains fish oil, garlic, rutin, and capsaicin.

Hsia et al., in U.S. Pat. No. 6,440,464, disclose a nutritive composition for cardiovascular heath comprising fish oil, garlic powder, rutin, capsaicin, vitamin A, vitamin C, vitamin E and one or more juice concentrates.

Policosanol is a name originally given to a unique extract of Cuban sugarcane (Saccharum officinarum) derived from the plant's waxy fraction. In 1964 the Cuban Institute of Research on Sugar Cane Derivatives wanted to identify high-value bioactive sugarcane derivatives. The first product with such potential was policosanol, sold in 40 countries as a patented agent for lowering cholesterol, with the exception of the United States as part of its continuing embargo on all things Cuban.

Policosanol is a mixture of many cosanols- eight long chain alcohols, in specific amounts.

Havana-based Dalmer Laboratories' Cuban policosanol product has been demonstrated to be effective in lowering cholesterol. Consequently, some companies have introduced imitations and false claims for the scientific efficacy of imitations of the Cuban original. Imitation policosanol products are produced from sugarcane wax extracts produced outside of Cuba, rice bran wax, and beeswax.

The beneficial side effects associated with Cuban policosanol supplementation include modestly reduced body weight, lowered blood pressure, decreased oxidative stress markers, and improved blood platelet functions. The side effects, including increased urination, headaches, dizziness and increased hunger, are less frequent than those accompanying statins.

Mas et al., reporting in Drugs R D 3(3): 159-172, 2002, noted that policosanol significantly decreased systolic blood pressure compared with baseline and placebo. In the study described, policosanol lowered serum low density lipoprotein cholesterol, total cholesterol, triglycerides, and the LDL-HDL ratio and total cholesterol-HDL ratio.

Famez et al., Drug RD 2005; 6(1); 11-19 treated rabbits with Policosanol and fish oil. Policosanol alone lowered LDL and total cholesterol, but left triglycerides unchanged. Combined therapy decreased LDL. Changes in total cholesterol, LDL and HDL with combined therapy were greater than with fatty acids, but similar to policosanol alone.

### SUMMARY OF THE INVENTION

It is an object of the present invention to overcome the aforesaid deficiencies in the prior art.

It is another object of the present invention to provide a composition to reduce low-density lipoproteins.

It is another object of the present invention to provide a composition to reduce triglycerides.

It is yet another object of the present invention to provide a composition to increase high-density lipoproteins.

It is still another object of the present invention to provide a composition that treats hypertension.

A composition that reduces triglycerides and low-density lipoprotein, while increasing high-density lipoprotein and alleviating hypertension, comprises a source of EPA and DHA, and at least one of oleuropein, allicin, and policosanol.

The compositions of the present invention comprise EPA and DHA in the form of free fatty acids; the ratio of EPA to DHA is preferably about 3:2 by weight, which is the ratio of EPA to DHA found in fish oil and algae. Of course, EPA and DHA from any source can be used in the composition.

The oleuropein is preferably obtained from olives, although any source of oleuropein can be used in compositions according to the present invention. Oleuropein is the bitter components of olives that is readily separated from the water layer formed when olive oil is separated for extraction, or by extracting the compound from olive leaves.

Allicin is preferably obtained from garlic. Allicin is produced by an enzymatic reaction when raw garlic is either crushed or is injured in some fashion. The enzyme allinase, stored in a separate compartment in the garlic, combines with allin in raw garlic to produce allicin. Of course, synthetic allicin can also be used.

Policosanol was a name originally given to a unique extract of Cuban sugarcane (Saccharum officinarum) derived from the waxy fraction of the plant. In 1954, the Cuban Institute of Research on Sugar Cane Derivatives identified high-value bioactive sugarcane derivatives including Policosanol. Policosanol is a mixture of higher primary aliphatic alcohols, the main component of which is octacosanol.

### DETAILED DESCRIPTION OF THE INVENTION

The compositions of the present invention can be used to treat hypertension as well as to treat elevated cholesterol and triglycerides. As shown by the data below, the combination of DHA and EPA and at least one of oleuropein, allicin, or policosanol reduces LDL and triglycerides, reduces blood pressure, and elevates HDL. The EPA and DHA, act synergistically with the oleuropein, allicin, or policosanol while the EPA and DHA retain their anti-inflammatory, anti-thrombotic and immunoregulatory properties while reducing LDL.

Eighty men with moderately elevated cholesterol who also suffered from moderate to high hypertension, were assigned to one of eight treatment groups, and followed for 16 weeks. The EPA and DHA administered was from fish oil and in free fatty acid form containing 3 grams of EPA and 2 grams of DHA, administered daily. Group one received fish oil alone. Group two received the same amount of fish oil with 5 mg allicin dissolved in the fish oil. Group three received the same amount of fish oil with 100 mg oleuropein dissolved therein. Group four received the same amount of fish oil with 10 mg of Policosanol dissolved therein. Group five received soft gelatin capsules with sunflower oil rather than fish oil. Group six received 5 mg of allicin dissolved in sunflower oil. Group seven received 100 mg oleuropein dissolved in sunflower oil. Group eight received Policosanol dissolved in sunflower oil.

After sixteen weeks of treatment, the results were as follows:
1. Group one registered 5% lowering of serum triglycerides. There was no change in HDL cholesterol and an 8% increase in LDL. Systolic blood pressure dropped by average of 3% and diastolic blood pressure dropped by average of 2%.
2. Group two registered a 30% lowering of triglycerides, 12% decrease in LDL, and 8% increase in HDL. Both systolic and diastolic blood pressure dropped by an average of 10%
3. Group three registered a 25% lowering of serum triglycerides, 10% decrease of LDL, and 10% increase in HDL. Systolic pressure dropped by average of 12% and diastolic pressure dropped by average of 10%
4. Group four registered 20% lowering of serum triglycerides, 12% decrease of LDL, and 8% increase in HDL. Systolic and diastolic pressure dropped by average of 10%.
5. Group five had no change in any of the parameters tested.
6. Group six registered 3% lowering of serum triglycerides, 6% lowering of LDL and 2% increase of HDL. Sistolic and diastolic pressure dropped by average of 5%
7. Group seven registered 12% lowering of serum triglycerides and no significant change in LDL or HDL levels. Systolic blood pressure dropped by average of 7% and diastolic pressure dropped by average of 5%.
8. Group eight registered 10% lowering of serum triglycerids, 8% lowering of LDL, and 5% increase of HDL. There was substantial change of blood pressure.

The results of this study show a surprising synergistic effect of the combination of fish oil with even trace amounts of allicin oleuropein and policosanol.

Patients are administered compositions according to the present invention containing from about 1 to about 10 grams EPA, from about 0.5 to about 8 grams DHA, and from about 50 to about 200 mg oleuropein, about 0.5 to about 10 mg allicin, and/or from about 1 to about 15 mg of policosanol. This amount is administered daily. It is preferred that the amount of EPA be slightly higher than the amount of DHA.

Pharmaceutical compositions according to the present invention can be administered by any convenient route, including parenteral, subcutaneous, intravenous, intramuscular, or transdermal. Preferably, administration may be by the oral route. The dosage administered depends upon the age, heath, and weight of the recipient, nature of concurrent treatment, if any, and the nature of the effect desired.

Compositions within the scope of the present invention include all compositions wherein the active ingredients are contained in an amount effective to achieve the intended purpose. While individual needs vary, determination of optimal ranges of effective amounts of each compound is within the skill of the art. Typical dosages comprise 0.01 to 100 mg/kg body weight. The preferred dosages comprising 0.1 to 100 mg/kg body weight. The most preferred dosages comprise 1 to 50 mg/kg body weight.

Pharmaceutical compositions for administering the active ingredients of the present invention may contain, in addition to the pharmacologically active combination of compounds, suitable pharmaceutically acceptable carriers comprising excipients and auxiliaries, which facilitate processing of the active compounds into preparations, which can be used pharmaceutically. Preferably, the preparations, particularly those preparations which are administered orally and which can be used for the preferred type of administration, such as tablets, dragees, and capsules, and also preparations which can be administered rectally, such as suppositories, as well as suitable solutions for administration by injection or orally, contain from about 0.01 to about 99 percent by weight, preferably from about 20 to 75 percent by weight, active compounds, together with the excipient. For purposes of the present invention, all percentages are by weight unless otherwise indicated. In addition to the following described pharmaceutical composition, the compounds of the present invention can be formulated as inclusion complexes, such as cyclodextrin inclusion complexes.

The pharmaceutically acceptable carriers include vehicles, adjuvants, excipients, or diluents that are well known to those skilled in the art and which are readily available. It is preferred that the pharmaceutically acceptable carrier be one which is chemically inert to the active compounds and which has no detrimental side effects or toxicity under the conditions of use.

The choice of carrier is determined partly by the particular active ingredient, as well as by the particular method used to administer the composition. Accordingly, there is a wide variety of suitable formulations of the pharmaceutical compositions of the present invention. Formulations can be prepared for oral, aerosol, parenteral, subcutaneous, intravenous, intra arterial, intramuscular, intra peritoneal, intra tracheal, rectal, and vaginal administration.

Suitable excipients are, in particular, fillers such as saccharides, for example, lactose or sucrose, mannitol or sorbitol, cellulose preparations and/or calcium phosphates, for example, tricalcium phosphate or calcium hydrogen phosphate, as well as binders such as starch paste using, for example, maize starch, wheat starch, rice starch, potato starch, gelatin, tragacanth, methyl cellulose, hydroxypropylmethylcellulose, sodium carboxymethylcelullose, and/or polyvinyl pyrrolidone.

Suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Optionally, the suspension may also contain stabilizers.

Other pharmaceutically acceptable carriers for the active ingredients according to the present invention are liposomes, pharmaceutical compositions in which the active ingredient is contained either dispersed or variously present in corpuscles consisting of aqueous concentric layers adherent to lipid layers. The active ingredients may be present both in the aqueous layer and in the lipid layer, inside or outside, or, in any event, in the nonhomogeneous system generally known as a liposomic suspension.

The hydrophobic layer, or lipid layer, generally, but not exclusively, comprises phospholipids such as lecithin and sphingomyelin, steroids such as cholesterol, more or less ionic surface active substances such as dicetyl phosphate, stearylamine, or phosphatidic acid, and/or other materials of a hydrophobic nature.

The compounds may also be formulated for transdermal administration, for example in the form of transdermal patches so as to achieve systemic administration.

Formulations suitable for oral administration can consists of liquid solutions such as effective amounts of the compounds emulsified in diluents such as water, saline, or orange juice; capsules, tables, sachets, lozenges, and troches, each containing a predetermined amount of the active ingredients as solids or granules; powders, suspensions in an appropriate liquid; and suitable emulsions. Liquid formulations may include diluents such as water and alcohols, e.g., ethanol, benzyl alcohol, and the polyethylene alcohols, either with or without the addition of a pharmaceutically acceptable surfactant, suspending agents, or emulsifying agents. Capsule forms can be of the ordinary hard- or soft-shelled gelatin type containing, for example, surfactants, lubricant, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscaramellose sodium, talc, magnesium stearate, calcium stearate, zinc stearate, stearic acid, and other preservatives, flavoring agents, and pharmaceutically acceptable disintegrating agents, moistening agents preservatives flavoring agents, and pharmacologically compatible carriers. Lozenge forms can comprise the active ingredient in a carrier, usually sucrose and acacia or tragacanth, as well as pastilles comprising the active ingredient in an inert base such as gelatin or glycerin, or sucrose and acacia. Emulsions and the like can contain, in addition to the active ingredient, such carriers as are known in the art.

Oils which can be used in parenteral formulations include petroleum, animal, vegetable, or synthetic oils. Specific examples of oils include peanut, soybean, sesame, cottonseed, corn, olive, petrolatum, and mineral. Fatty acids can be used in parenteral formulations, including oleic acid, stearic acid, and isostearic acid. Ethyl oleate and isopropyl myristate are examples of suitable fatty acid esters. Suitable salts for use in parenteral formulations include fatty alkali metal, ammonium, and triethanolamine salts, and suitable detergents include cationic detergents such as dimethyl dialkyl ammonium halides, and alkyl pyridimium halides; anionic detergents such as dimethyl olefin sulfonates, alkyl, olefin, ether, and monoglyceride sulfates and sulfosuccinates; polyoxyethylenepolypropylene copolymers; amphoteric detergents such s alkyl-beta-aminopropionates and 2-alkyl-imidazoline quaternarry ammonium salts; and mixtures thereof.

Parenteral formulations typically contain from about 0.5 to 25% by weight of the active ingredients in solution. Suitable preservatives and buffers can be used in these formulations. In order to minimize or eliminate irritation at the site of injection, these compositions may contain one or more nonionic surfactants having a hydrophilic-lipophilic balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be present in unit dose or multiple dose sealed containers, such as ampules and vials, and can be stored in a freeze-dried (lyophilized) condition requiring only the addition of the sterile liquid carrier, e.g., water, for injections immediately prior to use. Extemporaneous injection solutions and suspensions can be prepared from sterile powders, granules, and tablets of the kind previously described.

Additionally, the active ingredients can be formulated into suppositories by mixing the active ingredients with a variety of bases, including emulsifying bases or water-soluble bases. Formulations suitable for vaginal administration may be in the form of pessaries, tampons, creams, gels, pastes, foam, or spray formulations containing, in addition to the active ingredients, such carriers as are known in the art to be appropriate.

It is to be understood that the phraseology or terminology employed herein is for the purpose of description and not of limitation. The means and materials for carrying out various disclosed functions may take a variety of alternative forms without departing from the invention.

Thus, the expressions “means to . . . ” and means for . . . ” as may be found in the specification above and/or in the claims below, followed by a functional statement, are intended to define and cover whatever structural, physical, chemical, or electrical, element or structures which may now or in the future exist for carrying out the recited function, whether or nor precisely equivalent to the embodiment or embodiments disclosed in the specification above. It is intended that such expressions be given their broadest interpretation.

What is claimed is:

1. A composition for treating elevated low-density lipoprotein and triglycerides, low high-density lipoprotein and hypertension comprising an effective amount of a combination of EPA, DHA, and at least one compound selected from the group consisting of oleuropein, allicin, and policosanol.

2. The composition according to claim 1 wherein the EPA and DHA are obtained from fish oils or algae.

3. The composition according to claim 1 wherein the composition contains from about 1 to about 10 grams EPA, from about 0.5 to about 8 grams DHA, and from about 0.5 to about 250 mgs of at least one compound selected from the group consisting of oleuropein, allicin, and policosanol.

4. The composition according to claim 3 wherein the EPA is present in an amount of about 3 grams, the DHA is present in an amount of about 2 grams, and the at least one compound is present in an amount of about 0.5-250 mg.

5. The composition according to claim 1 wherein the compound is oleuropein.

6. The composition according to claim 1 wherein the compound is allicin.

7. The composition according to claim 1 wherein the compound is policosanol.

8. A method for treating elevated low-density lipoprotein, hypertension, elevated triglycerides and for increasing high-density lipoprotein comprising administering to a patient in need thereof a composition comprising an effective amount of a combination of EPA, DHA, and at least one of oleuropein, allicin, and policosanol.

9. The method according to claim 8 wherein the composition contains from about 1 to about 10 grams EPA, from about 0.5 to about 8 grams DHA, and from about 0.5 to about 250 mg of the at least one compound.

10. The method according to claim 8 wherein the compound is oleuropein.

11. The method according to claim 8 wherein the compound is allicin.

12. The method according to claim 8 wherein the compound is policosanol.

13. The method according to claim 8 wherein the EPA and DHA are obtained from fish oil or algae.
